(19) United States
(12) Patent Application Publication
(10) Pub. No.: US 2025/0049608 A1
(43) Pub. Date: Feb. 13, 2025

(54) Design of Disposable Urine Male pads

(71) Applicant: Mohaned Gaffer Mohammed Al-khamri, Aden (YE)

(72) Inventor: Mohaned Gaffer Mohammed Al-khamri, Aden (YE)

(21) Appl. No.: 18/782,327

(22) Filed: Jul. 24, 2024

(51) Int. Cl. A61F 5/44

(52) U.S. Cl. CPC A61F 5/4401

(57) ABSTRACT

A design for disposable Urine Male Pads (UMP) is presented. The male pad aims to absorb any urinary leakage. The male pad is designed to target a large section of the population including males with urinary incontinence diseases, stricture urethra and males with disabilities. The male pad comprises a cylindrical tube made of thin and flexible silicon layers overlaid with a thin layer of gelatinous tissue that is able to absorbed urine. The male pad is clipped to a male underwear and tight around the pelvis such that it is easily disposed and replaced with a new pad when necessary.

### BACKGROUND

The present disclosure is related to a disposable pad for males.

Urinary incontinence is characterized as an involuntary leakage of urine. Urine incontinence diseases has a large effect on a large section of the male population that limit their activities and lower their quality of life. Urinary incontinence causes include old age, enlarged prostates, stricture urethra and males with disabilities.

There is a need for disposable urine male pads (UMP) to help affected males of the population to lead a normal life.

### BRIEF SUMMARY

Urinary incontinence diseases has a huge toll on effected people's quality of life. Such diseases has a psychological, social and financial implications on affected people. Affected people tend to feel depressed, shame and insecurity, which hinders their daily social and work activities. As a result, they tend to isolate themselves, always need to be close to a toilet and always worried when participating in work related or social meetings. Furthermore, the cost of incontinence aid, which can be quite expensive.

A Urine Male Pad is designed to increase the quality of life by not limiting the activities of men with and enable Men with urinary incontinence diseases. The Urine Male Pad is designed to have to the following characteristics: simple to wear, light (not heavy), flexible, enable user to move freely, urine move freely with equal and even distribution of urine to the whole pad. The design utilizes certain amount of absorbent substance (crystal or gel) to absorb urine, e.g. gram of crystal, ml of gel, or both, that fill the UMP chamber(s), which in turn absorb liters of urine during a period of time that last up to 24 hour.

The UMP comprises two parts: a main part and a secondary part. The main part is easily applied by hanging and wrapping the main part around pelvis. The main part is flexibly worn by men with urine incontinence when they go about their daily activities, work meeting, travel, sleep, etc., with the flexibility of the user easily replacing the UMP with a new one without completely taking off the user's lower clothing. The secondary part is an additional part that is flexibly connected to the main part and wrapped around the users thighs so that it can be worn for longer duration and can accept higher amount of urine for those with disabilities or special needs. This part can be easily connected to the main part without potential urine leakage and this part should be connected to the main part at home.

### BRIEF DESCRIPTION OF THE DRAWING

FIG. 1: Design for a main part of a disposable Urine Male Pads (UMP)—Not to scale.

FIG. 2: Design for a secondary part of a disposable Urine Male Pads (UMP)—Not to scale.

### DETAILED DESCRIPTION

Urinary incontinence diseases has a huge toll on effected people's quality of life. Such diseases has a psychological, social and financial implications on affected people. Affected people tend to feel shame and insecurity, which hinders their daily social and work activities. As a result, they tend to isolate themselves, always need to be close to a toilet and always worried when participating in work related or social meetings.

A Urine Male Pad is designed to increase the quality of life by not limiting the activities of men with and enable Men with urinary incontinence diseases. The Urine Male Pad is designed to have to the following characteristics: simple to wear, light (not heavy), flexible, enable user to move freely, urine move freely with equal and even distribution of urine to the whole pad. The design utilizes certain amount of absorbent substance (crystal or gel) to absorb urine, e.g. gram of crystal, ml of gel, or both, that fill the UMP chamber(s), which in turn absorb liters of urine during a period of time that last up to 24 hour.

The UMP comprises two parts: a main part and a secondary part. The main part is easily applied by hanging and wrapping the main part around pelvis. The main part is flexibly worn by men with urine incontinence when they go about their daily activities, work meeting, travel, sleep, etc., with the flexibility of the user easily replacing the UMP with a new one without completely taking off the user's lower clothing. The secondary part is an additional part that is flexibly connected to the main part and wrapped around the users thighs so that it can be worn for longer duration and can accept higher amount of urine for those with disabilities or special needs. The secondary part can be easily connected to the main part without potential urine leakage and this part should be connected to the main part at home.

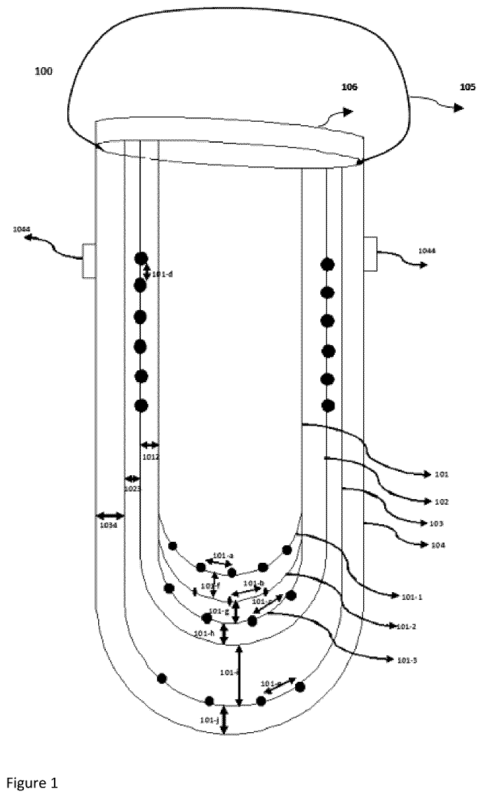

FIG. 1 illustrates the main part 100 of the UMP, referred hereinafter as a first part. FIG. 1 illustrates the disposable, one-use, first part 100. The disposable, one-use, first part comprising: a flexible and water resistant cylindrical shape part having an open first end and a closed second end, the open first end of the cylindrical shape part is configured to receive a male penis, wherein a male penis head reaches an interior of the closed second end, and a male penis base reaches an interior of the open part, wherein the open first end is made with an elastic material 106, wherein the open first end connects, through two holes, with a band, 105 in FIG. 1, worn around the male pelvis, where the band 105 can be connected to the open first end, by any means obvious to one skilled in the art, one no limiting example, is using a snap on buttons, which enable the user to wrap around the band and connect to the open first end with ease and without having to take off the user's clothing.

The cylindrical shape part is made in part of silicon and composed of multilayers, comprising:
(1) first inner most layer 101, coated with gel absorbent substance, consisting of non-allergic cotton layer and comprising a first dome-shape sub-layer 101-1, a second dome-shape sub-layer 101-2, and a third dome-shape sub-layer 101-3, placed at the interior of the closed second end to surround the received penis head, wherein each of the first dome-shape sub-layer, second dome-shape sub-layer and third dome-shape sub-layer comprises a first predetermined number of holes having a first diameter size, second predetermined number of holes having a second diameter size, and third predetermined number of holes having a third diameter size, respectively, wherein the holes on each first dome-shape sub-layer, second dome-shape sub-layer and third dome-shape sub-layer are spaced apart by a first predetermined hole distance 101-a, a second predetermined hole distance 101-b, and third predetermined hole distance 101-c, respectively, wherein the first dome-shape sub-layer and the second dome-shape sub-layer are spaced apart by a first predetermined dome-shape distance 101-f, the second dome-shape sub-layer and the third dome-shape sub-layer are spaced apart by a second predetermined dome-shape distance 101-g, where spacings between the 3 dome-shape sub-layers are coated with a combination of gel and crystal material having a thickness proportional to the spacings, such that urine not absorbed, is received in an order of first by the first dome-shape sub-layer, second dome-shape sub-layer, then the third dome-shape sub-layer, respectively,
(2) second inner most layer 102, coated with gel absorbent substance, adjacent to the first inner most layer, receiving, through the predetermined number of holes of the third dome-shape sub-layer, urine that has not been absorbed by the third dome-shape sub-layer, wherein the second inner most layer, comprising a fourth dome-shape sub-layer placed at the interior of the closed second end to surround the third dome-shape sub-layer and spaced apart from the third dome-shape sub-layer by a third predetermined dome-shape distance 101-h, and further comprising fourth predetermined number of holes having a fourth diameter size placed one third away from the open first end and two third away from the closed second end, wherein the fourth predetermined number of holes of the second inner most layer are spaced apart by a fourth predetermined hole distance 101d,
(3) third inner most layer, coated with gel absorbent substance, adjacent to the second inner most layer, receiving, through the fourth predetermined number of holes of the second inner most layer, urine that has not been absorbed by the second inner most layer, wherein the third inner most layer, comprising a fifth dome-shape sub-layer placed at the interior of the closed second end to surround the fourth dome-shape and spaced apart from the fourth dome-shape sub-layer by a fourth predetermined dome-shape distance 101-i, wherein the fifth dome-shape sub-layer comprises a fifth predetermined number of holes having a fifth diameter size, wherein the fifth predetermined number of holes of the third inner most layer are spaced apart by a fifth predetermined hole distance 101-e,
(4) fourth inner most layer, coated with gel absorbent substance, adjacent to the third inner most layer, receiving, through fifth predetermined number of holes of the fifth dome-shape sub-layer, urine that has not been absorbed by the fifth dome-shape sub-layer, wherein the fourth inner most layer comprising a dixth dome-shape sub-layer placed at the interior of the closed second end to surround the fifth dome-shape and spaced apart from the fifth dome-shape sub-layer by a fifth predetermined dome-shape distance 101-j, and further comprising 2 silicon rubber hole plug Snap 1044 placed one third away from the open first end and two third away from the closed second end, wherein the Silicone Rubber Hole Plug Snap connects to the second part 200 when required by the male user or career of the male user.

The first predetermined number of holes is larger than the second predetermined number of holes and the third predetermined number of holes; the third predetermined number of holes is larger than the second predetermined number of holes and the first predetermined number of holes; the fourth predetermined number of holes is larger than the first predetermined number of holes; and the fifth predetermined number of holes is smaller than the fourth predetermined number of holes and larger than the second predetermined number of holes.

the diameter of the holes in the first dome-shape sub-layer is larger than the diameter of the holes in the second dome-shape sub-layer; the diameter of the holes in the third dome-shape sub-layer is larger than the diameter of the holes in the first dome-shape sub-layer and the second dome-shape sub-layer; the diameter of the holes in the second inner most layer is larger than the diameter of the holes in the first dome-shape sub-layer, the second dome-shape sub-layer, and the third dome-shape sub-layer; and the diameter of the holes in the fifth dome-shape sub-layer is smaller than the diameter of the holes in the second inner most layer and larger than diameters of the holes in the second dome-shape sub-layer.

The second predetermined dome-shape distance 101-g is larger than the first predetermined dome-shape distance 101-f.

the first inner most layer and the second inner most layer are spaced apart by a first layer distance 1012; the second inner most layer and the third inner most layer are spaced apart by a second layer distance 1023; the third inner most layer and the fourth inner most layer are spaced apart by a third layer distance 1034; the first layer distance 1012 is larger than the second layer distance 1023 and the third layer distance 1034; and the third layer distance 1034 is larger than the second layer distance 1023.

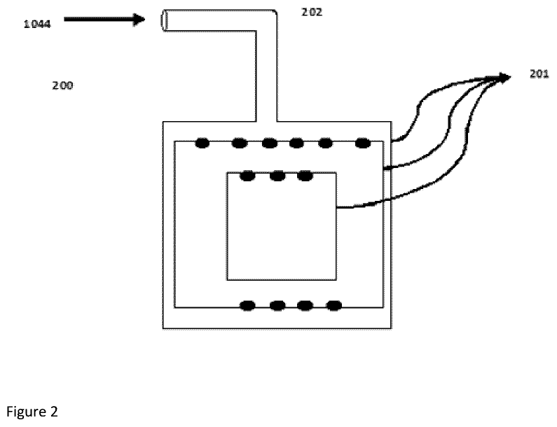

FIG. 2 illustrates an example, non-limiting, of a secondary part 200 of the UMP where urine is received from 1044 through an L-shaped tube 202, where 201 illustrates the multi-layer design with spaced apart holes in different layers. One design that produced optimal results is a design that is similar to the design of the main part of the UMP in FIG. 1, with the modification of a larger volume size.

the first part of the UMP further comprises a first color changing material and first beeping sound indicating the first part is full and to be replaced, and the second part of the UMP further comprises a second color changing material and second beeping sound, different from the first beeping sound, indicating the second part is full and to be replaced.

The design illustrated in FIG. 1, havening a different distance value for each parameter illustrated in FIG. 1, is an optimized design producing optimal results, resulting from a number of various and repeated designs implemented while taking into account the average number of urination times for average size males with urinary incontinence diseases in a span of 24 hours, and the volume of urine accumulated in a span of 24 hours.

1. A Urine Male Pad (UMP) for a male user comprising:
a disposable, one-use, first part, and a disposable, one-use, second part;
the disposable, one-use, first part comprising:
a flexible and water resistant cylindrical shape part having an open first end and a closed second end, the open first end of the cylindrical shape part is configured to receive a male penis, wherein a male penis head reaches an interior of the closed second end, and a male penis base reaches an interior of the open part, wherein the open first end is made with an elastic material, wherein the open first end connects, through two holes, with a band worn around the male pelvis;
the cylindrical shape part is made in part of silicon and composed of multilayers, comprising
first inner most layer, coated with gel absorbent substance, consisting of non-allergic cotton layer and comprising a first dome-shape sub-layer, a second dome-shape sub-layer, and a third dome-shape sub-layer, placed at the interior of the closed second end to surround the received penis head, wherein each of the first dome-shape sub-layer, second dome-shape sub-layer and third dome-shape sub-layer comprises a first predetermined number of holes having a first diameter size, second predetermined number of holes having a second diameter size, and third predetermined number of holes having a third diameter size, respectively, wherein the holes on each first dome-shape sub-layer, second dome-shape sub-layer and third dome-shape sub-layer are spaced apart by a first predetermined hole distance, a second predetermined hole distance, and third predetermined hole distance, respectively,
wherein the first dome-shape sub-layer and the second dome-shape sub-layer are spaced apart by a first predetermined dome-shape distance, the second dome-shape sub-layer and the third dome-shape sub-layer are spaced apart by a second predetermined dome-shape distance, where spacing's between the 3 dome-shape sub-layers are coated with a combination of gel and crystal material having a thickness proportional to the spacings, such that urine not absorbed, is received in an order of first by the first dome-shape sub-layer, second dome-shape sub-layer, then the third dome-shape sub-layer, respectively,
second inner most layer, coated with gel absorbent substance, adjacent to the first inner most layer, receiving, through the predetermined number of holes of the third dome-shape sub-layer, urine that has not been absorbed by the third dome-shape sub-layer, wherein the second inner most layer, comprising a fourth dome-shape sub-layer placed at the interior of the closed second end to surround the third dome-shape sub-layer, and further comprising fourth predetermined number of holes having a fourth diameter size placed one third away from the open first end and two third away from the closed second end, wherein the fourth predetermined number of holes of the second inner most layer are spaced apart by a fourth predetermined hole distance,
third inner most layer, coated with gel absorbent substance, adjacent to the second inner most layer, receiving, through the fourth predetermined number of holes of the second inner most layer, urine that has not been absorbed by the second inner most layer, wherein the third inner most layer, comprising a fifth dome-shape sub-layer placed at the interior of the closed second end to surround the fourth dome-shape, wherein the fifth dome-shape sub-layer comprises a fifth predetermined number of holes having a fifth diameter size, wherein the fifth predetermined number of holes of the third inner most layer are spaced apart by a fifth predetermined hole distance,
fourth inner most layer, coated with gel absorbent substance, adjacent to the third inner most layer, receiving, through fifth predetermined number of holes of the fifth dome-shape sub-layer, urine that has not been absorbed by the fifth dome-shape sub-layer, wherein the fourth inner most layer, comprising 2 silicon rubber hole plug Snap placed one third away from the open first end and two third away from the closed second end, wherein the Silicone Rubber Hole Plug Snap connects to the second part when required by the male user.

2. The Urine Male Pad (UMP) of claim 1, wherein:
the first predetermined number of holes is larger than the second predetermined number of holes and the third predetermined number of holes;
the third predetermined number of holes is larger than the second predetermined number of holes;
the fourth predetermined number of holes is larger than the first predetermined number of holes; and
the fifth predetermined number of holes is smaller than the fourth predetermined number of holes and larger than the second predetermined number of holes.

3. The Urine Male Pad (UMP) of claim 2, wherein:
the diameter of the holes in the first dome-shape sub-layer is larger than the diameter of the holes in the second dome-shape sub-layer;
the diameter of the holes in the third dome-shape sub-layer is larger than the diameter of the holes in the first dome-shape sub-layer and the second dome-shape sub-layer;
the diameter of the holes in the second inner most layer is larger than the diameter of the holes in the first dome-shape sub-layer, the second dome-shape sub-layer, and the third dome-shape sub-layer; and
the diameter of the holes in the fifth dome-shape sub-layer is smaller than the diameter of the holes in the second inner most layer and larger than diameters of the holes in the second dome-shape sub-layer.

4. The Urine Male Pad (UMP) of claim 3, wherein: the second predetermined dome-shape distance is larger than the first predetermined dome-shape distance.

5. The Urine Male Pad (UMP) of claim 4, wherein:
the first inner most layer and the second inner most layer are spaced apart by a first layer distance;
the second inner most layer and the third inner most layer are spaced apart by a second layer distance;
the third inner most layer and the fourth inner most layer are spaced apart by a third layer distance;
the first layer distance is larger than the second layer distance and the third layer distance; and
the third layer distance is larger than the second layer distance.

6. The Urine Male Pad (UMP) of claim 5, wherein:
the first part of the UMP further comprises a first color changing material and first beeping sound indicating the first part is full and to be replaced, and
the second part of the UMP further comprises a second color changing material and second beeping sound, different from the first beeping sound, indicating the second part is full and to be replaced.
